(19) United States
(12) Patent Application Publication
(10) Pub. No.: US 2014/0243826 A1
(43) Pub. Date: Aug. 28, 2014

(54) METHOD FOR CONNECTING FRACTURED BONE

(71) Applicant: Yechiel Gotfried, Kiryat Motzkin (IL)

(72) Inventor: Yechiel Gotfried, Kiryat Motzkin (IL)

(21) Appl. No.: 14/190,749

(22) Filed: Feb. 26, 2014

Related U.S. Application Data
(60) Provisional application No. 61/770,604, filed on Feb. 28, 2013.

(51) Int. Cl. A61B 17/88; A61B 17/72; A61B 17/80

(52) U.S. Cl. CPC A61B 17/8875; A61B 17/8052; A61B 17/7233
USPC 606/62; 606/104; 606/281

(57) ABSTRACT

Method to attach two sections of bone together in which a telescoping screw system is positioned through a hole of a fixation device attached to a first section of bone while the screw system is in a closed configuration in which the screw body is telescoped inside the screw barrel. The screw body is then advanced relative to the screw barrel to cause penetration of the forward, threaded end of the screw body into a second section of bone, and thereby bring the screw system toward an open configuration in which the screw body is telescoped outside the screw barrel. The screw barrel is also engaged to the fixation device via a cooperating engagement mechanism.

### CROSS REFERENCE TO RELATED APPLICATION

This application claims priority under 35 U.S.C. §119(e) of U.S. provisional patent application Ser. No. 61/770,604 filed Feb. 28, 2013, which is incorporated by reference herein.

### FIELD OF THE INVENTION

The present invention relates generally to a method for connecting a fractured bone and more specifically to a method for coupling bone portions across a fracture.

### BACKGROUND OF THE INVENTION

Plates and Intramedullary (IM) nails are devices used to stabilize fractures and allow for bone healing. Plates are typically attached to a fractured bone while IM nails are usually inserted into the medullary canal of the long bones of anatomical extremities, e.g., the femur or tibia. Both plates and IM nails have a region that generally includes at least one hole, transverse to the longitudinal axis of the plate or nail, for receiving an anchoring system, such as a screw, to connect and hence stabilize two parts of a fractured bone.

Many times, especially in osteoporotic bone, the bone is not pre-drilled, aside from the bone entry hole, and thus cannot easily accept a screw. As such, the screw used to couple bone across a fracture is provided with a self-cutting threaded end. When using a telescoping screw system, which passes through the transverse nail or plate hole, it is rather demanding to lock the screw barrel to the plate or nail at the time where the threaded end of the screw is advanced and screwed into the bone. One reason for this difficulty is because the screw barrel and the locking mechanism, i.e. male projection, may deviate from the female counterpart notch.

### OBJECTS AND SUMMARY OF THE INVENTION

An object of an embodiment in accordance with the invention is to provide a method to overcome this difficulty of locking a screw barrel to a plate or IM nail where the objective of the threaded end of a screw is to penetrate into bone.

A method to connect two sections of bone together in accordance with the invention using a telescoping screw system that is positioned through a hole of a fixation device positioned at a first section of bone while the screw system is in a closed configuration in which a screw body is telescoped inside a screw barrel. The screw barrel is engaged to the fixation device via a cooperating engagement mechanism. The screw body is then advanced relative to the screw barrel to cause penetration of the forward, threaded end of the screw body into a second section of bone, and thereby bring the screw system toward an open configuration in which the screw body is telescoped outside the screw barrel.

### BRIEF DESCRIPTION OF THE DRAWINGS

The invention, together with further objects and advantages thereof, may best be understood by reference to the following description taken in conjunction with the accompanying drawings, wherein like reference numerals identify like elements, and wherein:

FIG. 1A shows a telescoping screw system in accordance with the invention in a closed configuration in which a screw body is telescoping all the way inside a screw barrel;

FIG. 1B is a partial cross-sectional view of the portion of the screw system circled in FIG. 1A showing a polygonal-shaped bore at a rear back end of the screw body followed by a threaded recess;

FIG. 2 shows the screw system in an open configuration in which the screw body is telescoping, all the way outside of the screw barrel

FIG. 3 shows the telescoping screw system attached to a screw driver and in the closed configuration extending through a hole of a fixation device;

FIG. 4 shows the telescoping screw system of FIG. 3 but in the open configuration with the screw body telescoping out of the screw barrel, and the male projection locked inside the female counterpart; and

FIG. 5 is a front view of an IM nail showing female counterpart notches inside a nail hole, transverse to a longitudinal axis of the IM nail.

### DETAILED DESCRIPTION OF THE INVENTION

Referring to the accompanying drawings wherein like reference refer to the same or similar elements, a method for connecting a fractured bone in accordance with the invention involves use of a telescoping screw system 1. The telescoping screw system 1 includes an elongate screw body 2 and an elongate screw barrel 3 that overlies and surrounds a portion of the screw body 2. The axial length of the screw barrel 3 is less than the axial length of the screw body 2.

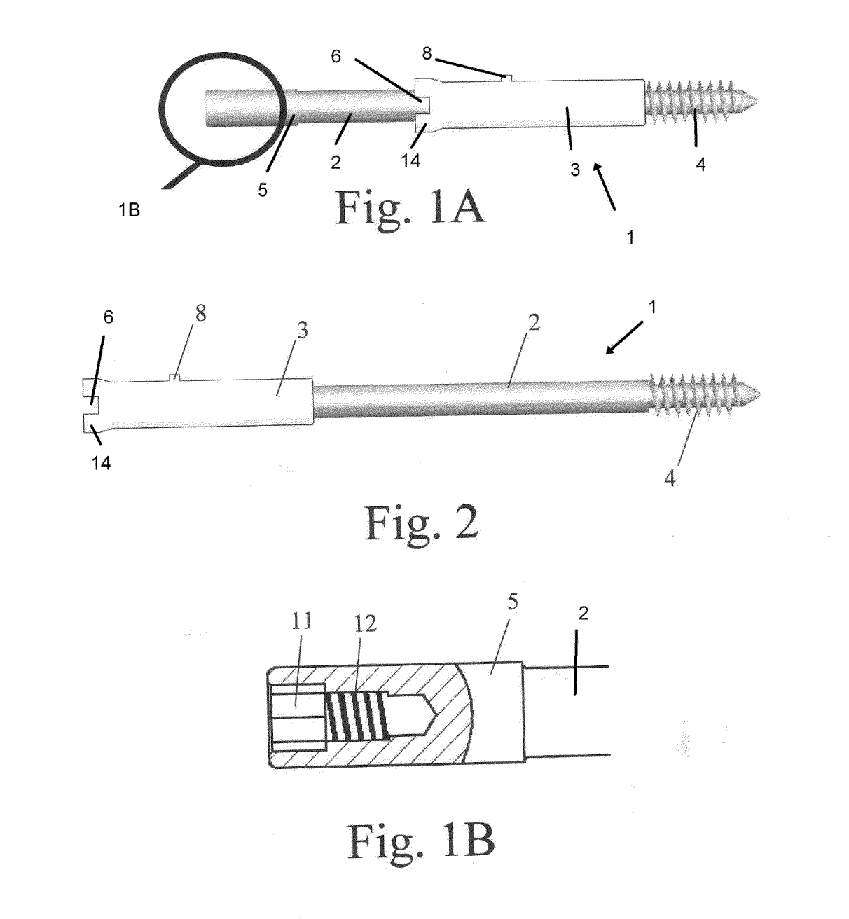

The screw body 2 has a substantially cylindrical main body portion, a front end having a thread 4 adapted to screw into bone upon rotation of the screw body 2, and a rear portion 5 that has a slightly larger diameter that the main body portion of the screw body 2 (see FIGS. 1A and 1B). Screw body 2 may be a unitary structure. The characteristics of the thread 4 on the screw body 2 may be appropriately determined by one skilled in the art to which the invention pertains with the objective of the thread 4 being to enable penetration of the screw body 2 into bone of a patient being treated by the method using the telescoping screw system 1.

At an opposite, rear end, the screw body 2 has a polygonal and threaded bore 11 (see FIG. 1B). A threaded recess 12 is formed at the rear end in communication with the bore 11. The specific polygonal shape of the bore 11 and threading and other characteristics of the recess 12 may vary based on, for example, the working end of a screwdriver 7 or other surgical instruments designed to interact with the screw body 2. A hexagonal shape of the bore 11 is provided in a preferred embodiment.

The screw barrel 3 has a substantially tubular form with a cylindrical interior passage. At least one male projection 8 is formed on the outer surface of the screw barrel 3. Although only one male projection 8 is shown, there may be several such male projections and of different shapes arranged at different locations on the outer surface of the screw barrel 3. Each male projection 8 is designed to engage a corresponding female counterpart structure 10 in or around a hole 15 of a fixation device 9, such as a plate on a bone or an IM nail in a bone (see FIG. 5). The female counterpart structure 10 may take any one of a number of different forms, such as a hollow, a slot, a notch or a groove as well as different geometrical configurations.

A rear end portion 14 of the screw barrel 3 flares outward slightly to provide the rear end with a larger diameter than a remaining portion of the screw barrel 3 (aside from the presence of the male projection 8). This flared rear end portion 14 includes one or more cut-outs or notches 6 that enable a screwdriver 7 or similar implement to engage with the screw body 2 without interference from the screw barrel 3 when the telescoping screw system 1 is in its open or closed configuration shown in FIGS. 1-4.

The screw body 2 is telescopically placed inside the screw barrel 3, i.e., the screw body 2 is movable along its longitudinal axis through the interior passage of the screw barrel 3 (in the directions represented by arrow A, see FIG. 1A)

The convention related to the telescoping screw system 1 is that it is open when the screw body 2 is telescoping all the way out of the screw barrel 3, i.e., the screw body 2 is urged all the way forward through the passage defined by the screw barrel 3 or the screw barrel 3 is pulled back, to assume the configuration shown in FIG. 2. The screw system 1 is closed when the screw body 2 is telescoping all the way inside the screw barrel 3, i.e., the screw body 2 is urged all the way to the rear through the passage defined by the screw barrel 3 or the screw barrel 3 is urged forward, to assume the configuration shown in FIG. 1A. In the closed configuration, the rear end of the screw body 2 may be located outside the screw barrel 3 as shown in FIG. 1A.

Various lengths of the screw body 2 may be provided in different telescoping screw systems 1. The particular length of screw body 2 to use in a specific situation may depend on, for example, the physical characteristics of the fracture of the bone and the position of the two bone sections being attached together.

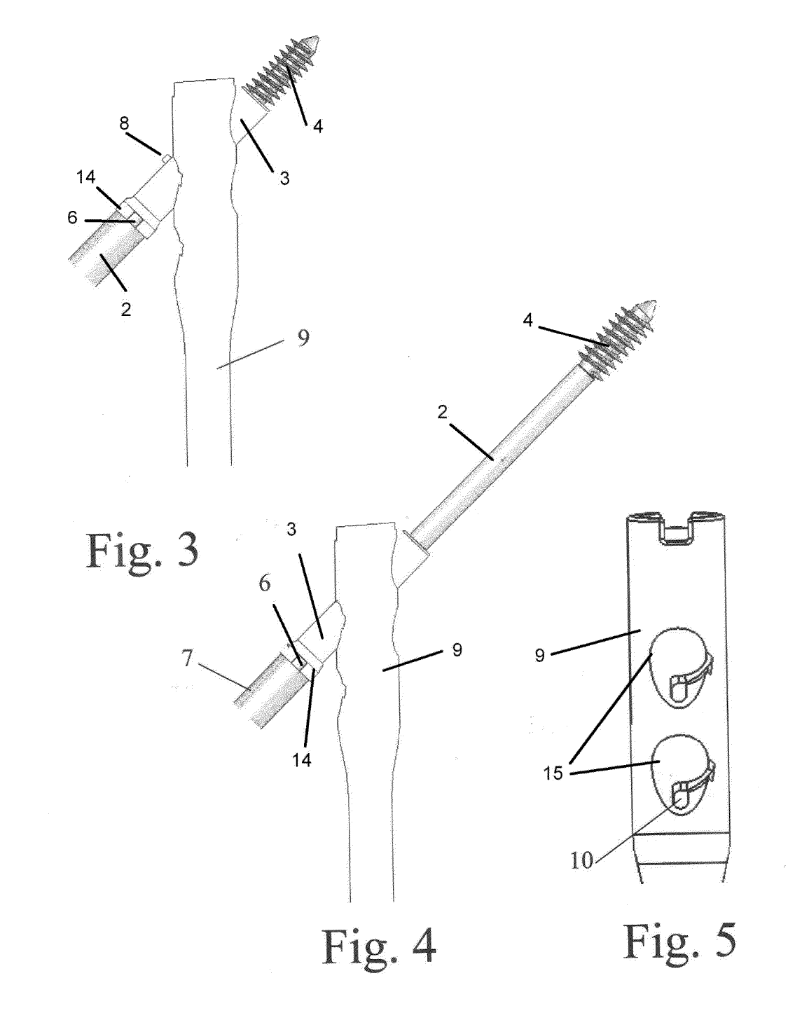

In an exemplifying, non-limiting method to use the telescoping screw system 1, two sections of bone are attached together, whether two sections of the same bone across a fracture of this bone or two different bones. The telescoping screw system is positioned through a hole 15 of a fixation device, e.g., nail 9, positioned at a first section of bone while the screw system 1 is in a closed configuration (FIG. 1A) in which the screw body 2 is telescoped inside the screw barrel 3 (as shown in FIG. 3). The screw barrel 3 is engaged to the fixation device via a cooperating engagement mechanism (male projection 8, notch 10). The screw body 2 is then advanced relative to the screw barrel 3 to cause penetration of the forward, threaded end of the screw body 2 into a second section of bone, and thereby bring the screw system 2 toward an open configuration in which the screw body 2 is telescoped fully outside the screw barrel 3 (see FIG. 4). Although full telescoping of the screw body 2 relative to the screw barrel 3 is mentioned, the telescoping is preferably a full telescoping but does not necessarily have to be a full telescoping.

Screwdriver 7 is attached to the barrel via cut outs 6 thereby advancing and rotating the screw barrel's male locking mechanism to engage a female counterpart at the fixation device.

Advancement of the screw body 2 relative to the screw barrel 3 may entail attaching an implement, e.g., a screwdriver, not shown, to the rear end 5 of the screw body 2 and actuating the implement, i.e., rotating the screwdriver. To aid in this step, the rear end 5 of the screw body 2 includes the polygonal recess or bore 11, into which the screwdriver is positioned. This screwdriver thus includes a mating end that mates with the polygonal recess or bore. To bring the second section of bone closer to the first section of bone, i.e., compress the bone fracture and aid in positioning the cooperating engagement mechanism, another screwdriver or implement is attached to the screw body 2 via the threaded recess 12, i.e., includes a threaded front end that mates with the threading of the recess 12. This enables the screw body 2 to be retracted in a direction opposite to a direction in which it is advanced. This screwdriver may be separated or disconnected from the rear end 5 of the screw body 2 after the screw barrel 3 is engaged to the fixation device, i.e., nail 9.

In one embodiment, the screw system 1 is attached to the screwdriver 7 before positioning the screw system 1 through the hole 15 of the fixation device. In this manner, the screw system 1 is positioned through the hole 15 of the fixation device while the screw system 1 is attached to the screwdriver 7.

Engagement of the screw barrel 3 to the fixation device via the cooperating engagement mechanism may entail positioning the screw barrel 3 to cause the male member 8 on the outer surface of the screw barrel 3 to enter into and lock in a female member, groove or notch 10, in or around the hole 15 of the fixation device. The screw barrel 3 is normally engaged to the fixation device before advancing the screw body 2 relative to the screw barrel 3 to cause penetration of the forward, threaded end of the screw body 2 into the second section of bone. This procedure can be performed in an opposite manner where licking of the screw barrel's male component to the fixation device's female component is performed after open telescoping of the screw into the second part of the bone.

Various constructions of the telescoping screw system 1 are also considered part of the invention.

While particular embodiments of the invention have been shown and described, it will be obvious to those skilled in the art that changes and modifications may be made without departing from the invention in its broader aspects, and, therefore, the aim in the appended claims is to cover all such changes and modifications as fall within the true spirit and scope of the invention.

I claim:

1. A method for attaching two sections of bone, comprising:
positioning a telescoping screw system through a hole of a fixation device positioned at a first section of bone while the screw system is in a closed configuration in which a screw body is telescoped inside a screw barrel, the screw body being movable axially in an interior passage of the screw barrel;
engaging the screw barrel to the fixation device via a cooperating engagement mechanism; and
advancing the screw body relative to the screw barrel to cause penetration of a forward end of the screw body into a second section of bone, and thereby bring the screw system toward an open configuration in which the screw body is telescoped outside the screw barrel.

2. The method of claim 1, wherein the step of advancing the screw body relative to the screw barrel comprises attaching an implement to a rear end of the screw body and actuating the implement.

3. The method of claim 1, wherein the step of advancing the screw body relative to the screw barrel comprises attaching a screwdriver to a rear end of the screw body and rotating the screwdriver.

4. The method of claim 3, wherein a rear end portion of the screw body includes a polygonal recess, the screwdriver engaging with the recess.

5. The method of claim 3, further comprising retracting the screwdriver in a direction opposite to a direction in which it is advanced and while attached to the screw body to thereby bring the second section of bone closer to the first section of bone.

6. The method of claim 5, further comprising separating the screwdriver from the rear end of the screw body after the screw barrel is engaged to the fixation device.

7. The method of claim 1, further comprising attaching the screw system to an implement before positioning the screw system through the hole of the fixation device, the screw system being positioned through the hole of the fixation device while the screw system is attached to the implement.

8. The method of claim 1, wherein the fixation device is a plate.

9. The method of claim 1, wherein the fixation device is an intramedullary nail.

10. The method of claim 1, wherein the step of engaging the screw barrel to the fixation device via a cooperating engagement mechanism comprises positioning the screw barrel to cause a male member on an outer surface of the screw barrel to enter into and lock in a female member in or around a hole of the fixation device.

11. The method of claim 1, wherein the step of engaging the screw barrel to the fixation device via a cooperating engagement mechanism comprises positioning the screw barrel to cause a projection on an outer surface of the screw barrel to enter into and lock in a groove in or around a hole of the fixation device.

12. The method of claim 1, wherein the screw barrel is engaged to the fixation device after advancing the screw body relative to the screw barrel to cause penetration of the forward end of the screw body into the second section of bone.

13. The method of claim 1, wherein the forward end of the screw body is threaded to enable it to penetrate into the second section of bone when advanced relative to the screw barrel.

14. A method for attaching two sections of bone, comprising:
positioning a fixation device at a first section of bone, the fixation device including at least one hole;
positioning a telescoping screw system through one of the at least one hole of the fixation device while the screw system is in a closed configuration in which a screw body is telescoped inside a screw barrel, the screw body being movable axially in an interior passage of the screw barrel;
engaging the screw barrel to the fixation device via a cooperating engagement mechanism; and
advancing the screw body relative to the screw barrel to cause penetration of a forward end of the screw body into a second section of bone, and thereby bring the screw system toward an open configuration in which the screw body is telescoped outside the screw barrel.

15. The method of claim 14, wherein the step of advancing the screw body relative to the screw barrel comprises attaching a screwdriver to a polygonal bore at the rear end of the screw body and rotating the screwdriver.

16. The method of claim 14, further comprising bringing the second section of bone closer to the first section of bone by attaching a screwdriver to a threaded recess at the rear end of the screw body and retracting the screwdriver in a direction opposite to a direction in which it is advanced and while attached to the screw body to thereby bring the second section of bone closer to the first section of bone.

17. The method of claim 14, further comprising attaching the screw system to a screwdriver before positioning the screw system through the hole of the fixation device, the screw system being positioned through the hole of the fixation device while the screw system is attached to the screwdriver.

18. The method of claim 14, wherein the fixation device is a plate.

19. The method of claim 14, wherein the fixation device is an intramedullary nail.

20. The method of claim 14, wherein the step of engaging the screw barrel to the fixation device via a cooperating engagement mechanism comprises positioning the screw barrel to cause at least one male member on an outer surface of the screw barrel to enter into and lock in a respective female member in or around a hole of the fixation device.
